(19) United States
(12) Patent Application Publication
(10) Pub. No.: US 2005/0209445 A1
(43) Pub. Date: Sep. 22, 2005

(54) Process for producing enriched fractions containing upto 100% of bacoside a and bacoside b from plant materials of bacopa species

(76) Inventors: Ganga Raju Gokaraju, Andhra Pradesh (IN); Rama Raju Gokaraju, Andhra Pradesh (IN); Venkata Subbaraju Gottumukkala, Andhra Pradesh (IN); Sridhar Pratha, Andhra Pradesh (IN)

Correspondence Address: RADER FISHMAN & GRAUER PLLC, LION BUILDING, 1233 20TH STREET N.W., SUITE 501, WASHINGTON, DC, 20036, US

(21) Appl. No.: 10/516,107

(22) PCT Filed:
(86) PCT No.: PCT/IN03/00002

(51) Int. Cl. C07J 17/00; C07J 71/00

(52) U.S. Cl. 536/6.1; 540/93; 424/725

(57) ABSTRACT

The invention relates to extraction concentration and separation of fractions containing Bacosides A and B from plant materials of Bacopa species. The dried plant materials are subjected to alcoholic or hydroalcoholic or water extraction and concentration of the extract. This solid mass is washed with a nonpolar organic solvent to remove fatty materials therefrom. The solid obtained thereafter is extracted with an organic polar solvent, the solvent extract is washed with water to remove water soluble contaminants therefrom and then dried in vacuum. This factor which is enriched with Bacosides A and B is subjected repeated chromatographic separation and purification to obtain Bacoside A and Bacoside B therefrom.

### TECHNICAL FIELD OF THE INVENTION

Analysis of traditional system of medicines such as Ayurveda and Sidda to identify natural and biological products that are potentially effective as curatives and prophylactics has emerged as a major approach in pharmaceutical research.

Infusion or extract of the plant Bacopa monniera Linn has been used for centuries in traditional Ayurvedic medicines as a memory enhancing and rejuvenating agent Bacopa monniera is known in Hindi and other local Indian lanugages as Brahmi and has been prescribed as a cure for mental disorders, weak intellect, anxiety and lack of concentration Anti-inflammatory, analgesic, antipyretic and antiepileptic properties of an extract of Brahmi have also been recorded.

Bacopa monniera Linn is from the family Scrophulariaceae and is a common creeping perennial herb that grows luxuriantly in moist, damp and marshy areas throughtout India and is commonly known as Brahmi or Jal Brahmi.

An alcoholic extract of Bacopa monniera has shown improved acquisition, consolidation and retention of newly acquired behavioral responses in albino rats. Major chemical constituents of the alcoholic extracts are Saponins. Bacoside A and Bacoside B. These saponins have been established as the components reponsible for memory enhancing and the like properties (Singh and Dhawan, Indian Journal of Pharmacology, 29, S 359-S 365, 1997; Singh, et al, Phytotherapy Research, 2, 70-75, 1988, published from the USA). A double-blind placebo controlled study carried out on 110 school children in the age group of 10-13 years to assess the effect of the extract of Bacopa monniera revealed significant enhancement in their arithmetic skills and memory (R Abhang, Journal of Research in Ayurveda and Sidda, 14, 10-24, 1993).

In a well designed double-blind, placebo controlled clinical study on 46 healthy human volunteers, Stough, et al have shown that Bacopa monniera extract improves higher order cognitive processes. (Psychopharmacology, 156, 481-484, 2001, published by Springer Verla Germany).

### BACKGROUND OF THE INVENTION

Alcoholic extract obtained by exacting a dried plant material is found to contain two fractions designated as Bacoside A and Bacoside B. Both the fractions have same aglycone moities and sugar residues. Haemolytic activity of Bacoside B is found to be much higher than that of Bacoside A. Bacosides A and B are crystalline mixtures of tritpeniod glycosides of the general formula I and II shown herein below. Bacoside A has been isolated in the pure state though Bacoside B has not yet been isolated in the pure state.

These compounds have either jujobogenin or pseudojujobogenin skeleton and contains one or more arabinose or glucose units.

[image]

[image]

### DISCLOSURE OF THE INVENTION

The present invention is aimed at enriching the Bacoside A and B concentration in the plant extract and further separation of the individual saponins therefrom. This objective is achieved by using a combination of solvents for preliminary extraction from the plant source and thereafter by chromatographic separation process.

Ethanolic or hydroethanolic extract obtained from dried plant material is dried preferably in vacuum. Fats and other contaminants are removed from the dry solid mass by treating the same from petroleum ether or hexane. The mass obtained after removal of fatty materials therefrom is extracted with a polar organic solvent selected from ethyl acetate, n-butyl acetate, ethyl propionate, isopropanol, n-butanol, n-pentanol or mixtures thereof. Organic layer containing Bacoisdes is separated, washed with water to remove undesired water soluble impurities such as sugars and salts, and then concentrated and dried under vacuum to obtain an enriched fraction containing Bacosides A and B in the range of 30 to 80% This enriched fraction is subjected to silica gel chromatography using as eluants, chloroform, methanol or water or a mixture of them to obtain a fraction containing 80%-100% of the Bacosides. Rechromatography of this enriched fraction results in the separation of individual Bacoside A and B. It is possible to carry out this chromatographic separation on a reversed phase silica gel.

This invention relates to a process for producing enriched fractions containing up to 100% of Bacoside A and Bacoside B from plant materials of Bacopa species which comprises the steps of subjecting plant materials of Bacopa species to organic solvent extraction, drying said extract, washing said dried mass with non-polar organic solvents to remove fatty materials therefrom, extracting the residue with at least one organic polar solvent washing said extract with water, and concentrating the solvent fraction under vacuum to obtain a concentrate containing 30 to 80% of Bacosides A and B, subjecting said concentrate to chromatographic separation and purification using organic solvents and water or mixtures thereof to obtain a faction containing 80 to 100% of Bacosides A and B and subjecting said enriched fraction to rechromatography to separate Bacoside A from Bacoside B therefrom. The last two chromatographic steps could also be combined into a single chromatographic separation.

This invention also includes Bacoside A and Bacoside B prepared and separated by the process described herein above.

The following examples disclose the best method of carrying out this invention.

### EXAMPLE 1

An alcoholic or hydroalcoholic extract is prepared in the conventional manner from dried plant materials of Bacopa monniera. After removing the solvents by evaporation preferably under vacuum a solid which contains a mixture of Bacoside A and Bacoside B is obtained This mixture containing 20 to 30% of the saponins is then extracted with petroleum ether three times to remove fatty materials therefrom. The solid obtained after petroleum ether extraction is refluxed with ethyl acetate at least twice. The combined solvent extract is washed with water and then dried over sodium sulphate initially and then concentrated and dried under vacuum to give a fraction containing 60 to 65% Bacosides A and B. This is then subjected to chromatographic separation over silica gel using chloroform and methanol as eluants to a faction containing 95% of Bacoside A and B. This is further chromatographed to separate the two fractions of the general formula I and II, into individual Bacosides A and B.

### EXAMPLE II

In this example, dried hydroalcoholic extract of Bacopa monniera containing 20 to 30% Bacosides A and B are repeatedly extracted with acetone and the combined acetone layer is concentrated under vacuum This is then treated with hexane and dried under vacuum to provide a fraction containing 59% of Bacosides A and B. This fraction is subjected to repeated chromatographic separation to separate Bacosides A and B of high purity.

Though these examples are with reference to Bacopa monniera, other plants belonging to the Bacopa species may be used. The saponin content may vary from species to species and will also depend on various other factors such as the state of growth of the plants.

Enriched fractions of Bacosides or the pure Bacosides separated by this method could be used in lower dosage for therapeutic use.

1. A process for producing enriched fractions containing upto 100% of Bacoside A and Bacoside B from plant materials of Bacopa species which comprises the steps of subjecting plant materials of Bacopa species to water or organic solvent extraction, drying said extract, washing said dried mass with non-polar organic solvents to remove fatty materials therefrom, extracting the residue with at least one organic polar solvent, washing said solvent extract with water, and containing said solvent fraction under vacuum to obtain a concentrate containing 30 to 80% of Bacosides A and B, subjecting said concentrate to chromatographic separation and purification using organic solvents and water or mixtures thereof as eluants to obtain a fraction containing 80 to 100% of Bacosides A and B and subjecting said enriched fraction to rechromatography to separate Bacon A from Bacoside B. The last two chromatography steps could also be combined to obtain individual Bacoside A and Bacoside B.

2. The process as claimed in claim 1, wherein the plant material is dried Bacopa monniera and the first solvent extraction of the dried plant material is carried out with ethyl alcohol or methyl alcohol or hydroalcohol or water.

3. The process as claimed in claim 2, wherein said solvent extract is evaporated and dried in vacuum.

4. The process as claimed in claim 1, wherein said non-polar solvent for removing fatty material from said solid plant extract is petroleum ether, hexane and the like.

5. The process as claimed in claim 1, wherein said organic polar solvent is selected from ethyl acetate, n-butyl acetate, acetone, ethyl propionate, n-pentanol, or n-butanol or combinations thereof to obtain an enriched fraction of Bacosides A and B in the organic layer.

6. The process as claimed in claim 5, wherein said organic layer is washed with water and then dried under vacuum to obtain a fraction containing 30 to 80% of the Bacosides.

7. The process as claimed in claim 1 wherein the first chromatographic separation is carried out on silica gel columns using chloroform, acetone, methanol, ethyl acetate, water or combinations thereof to obtain a fraction containing 80 to 100% of Bacosides A and B.

8. The process as claimed in claim 1, wherein said first chromatographic separation is carried out on reversed phase silica gel using acetonitrile, methanol, water or combinations thereof as eluants to obtain 80 to 100% of Bacosides A and B.

9. The process as claimed in claim 1, wherein the fraction enriched with Bacosides A and B are further separated by chromatography to obtain fractions containing pure Bacoside A and Bacoside B.

10. Bacoside A and Bacoside B when prepared by a process as claimed in any of the preceeding claims.
